(19) United States
(12) Patent Application Publication
(10) Pub. No.: US 2008/0275426 A1
(43) Pub. Date: Nov. 6, 2008

(54) Flexible and Durable Tip

(75) Inventors: Thomas J. HOLMAN, Minneapolis, MN (US); Tracee Eidenschink, Wayzata, MN (US); John J. Chen, Plymouth, MN (US); Daniel J. Horn, Shoreview, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/743,753

(22) Filed: May 3, 2007

(51) Int. Cl. A61M 25/16

(52) U.S. Cl. 604/523

(57) ABSTRACT

A method of making a flexible, durable catheter tip is achieved by providing a catheter tip, and in some embodiments, applying heat energy only to the distal portion of a catheter tip. In other embodiments, the method includes ablating a portion of the outer diameter of the proximal region material. In at least one embodiment, the method includes manufacturing a catheter tip with a layer of a harder material disposed about a first layer of a relatively softer material and then ablating at least a portion of the harder material. In some embodiments, the method includes dipping at least a portion of the distal region of the catheter tip into a chemical bath. In some embodiments, the method includes providing a catheter tip made of a tip material, adding a curable adhesive to at least a portion of the distal region, and then applying heat energy to cure the curable adhesive.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Catheters and catheter assemblies for use in medical procedures, such as angioplasty, stent delivery, etc. are well known. One aspect of the catheter performance is its “crossability.” Crossability is the ability to navigate the catheter across narrow restrictions in the vasculature. The characteristics of the tip of the catheter, located at the distal-most portion of the catheter, greatly affect the catheter's crossability. These characteristics include its profile, flex, and the balloon-to-tip transition. Catheter issues which affect crossability include catheter tips which do not bend sufficiently, catheter tips which protrude at undesirable angles, and catheter tips which flex and do not maintain their generally circular shape. These catheter issues may prevent the catheter from crossing a lesion. In the past, attempts to design tips in order to maximize crossability have created other concerns.

Although the problems identified above would seemingly be solved by using a softer material at the catheter tip, instead the softer material adversely affects the robustness of the tip. Decreased tip robustness often creates undesirable results such as the tip flaring when the balloon catheter is navigating a bend, or the catheter kinking at the region where the tip transitions to the balloon.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention, a brief summary of some of the claimed embodiments of the invention is set forth below. Additional details of the summarized embodiments of the invention and/or additional embodiments of the invention may be found below in the Detailed Description of the Invention. Also, a brief abstract of the technical disclosure in the specification is provided for the purposes of complying with 37 C.F.R. §1.72.

### BRIEF SUMMARY OF THE INVENTION

Embodiments of the invention contemplate a catheter tip and method of manufacturing same.

One embodiment of the present invention is directed to a method of making a flexible catheter tip. In some embodiments, the method includes applying heat energy only to the distal portion of a catheter tip, thereby modifying the hardness, crystallinity, and/or the thickness of the distal portion.

In at least one embodiment, the method of making the flexible tip includes ablating at least a portion of the outer diameter of the proximal region material, thereby making the distal region material thicker than the proximal region material.

In some embodiments, the method includes manufacturing a catheter tip with a layer of a harder material disposed about a first layer of a relatively softer material and then ablating at least a portion of the harder material of at least the proximal region of the catheter tip, thereby leaving the distal region material harder than the proximal region material.

In at least one embodiment, the method includes providing a catheter tip with a hardness and a crystallinity and then dipping at least a portion of the distal region of the catheter tip into a chemical bath, thereby changing the hardness and/or crystallinity of the distal region relative to the proximal region.

In some embodiments, the method includes providing a catheter tip made of a tip material, adding a second material to at least a portion of the distal region, and then applying heat energy or other catalyst to cure the curable adhesive.

These and other embodiments which characterize the invention are pointed out with particularity in the claims annexed hereto and forming a part hereof However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE DRAWINGS

A detailed description of the invention is hereafter described with specific reference being made to the drawings.

FIG. 1 is a cross-sectional view of a catheter with a catheter tip;

FIG. 2 is a partial cross-sectional view of an embodiment of the catheter tip of FIG. 1, illustrating the distal region of the tip during application of a heat source;

FIG. 3 is a partial cross-sectional view of an embodiment of the catheter tip of FIG. 1, illustrating the distal region of the tip before ablation;

FIG. 4 is a partial cross-sectional view of an embodiment of the catheter tip of FIG. 1, illustrating the distal region of the tip of FIG. 3 after ablation;

FIG. 5 is a partial cross-sectional view of an embodiment of the catheter tip of FIG. 1, illustrating a distal region of the tip with a hard layer and a soft layer;

FIG. 6 is a partial cross-sectional view of an embodiment of the catheter tip of FIG. 1, illustrating the distal region of the tip of FIG. 5 after a portion of the hard layer has been ablated;

FIG. 7 is a partial cross-sectional view of an embodiment of the catheter tip of FIG. 1, illustrating the distal region of the tip after application of a curable adhesive;

FIG. 8 is a partial cross-sectional view of an embodiment of the catheter tip of FIG. 1, illustrating the distal region of the tip being dipped in a chemical bath; and

FIG. 9 is a partial cross-sectional view of an embodiment of the catheter tip of FIG. 1, illustrating the distal region of the tip of FIG. 8 after being dipped in a chemical bath.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, and/or substituted for, elements depicted in another figure as desired.

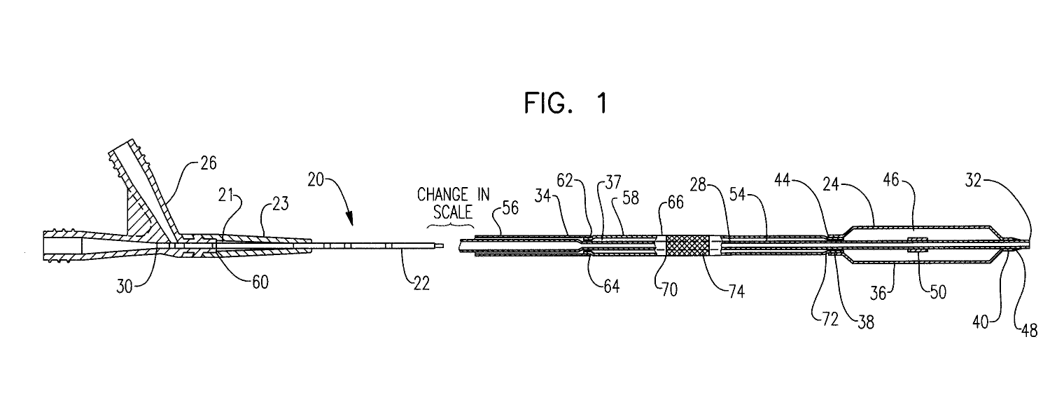

In at least one embodiment of the invention, an example of which is shown in FIG. 1, a catheter 20 is illustrated. In some embodiments the invention is directed to any type of catheter suitable for use in a medical procedure. For example, the catheter depicted in FIG. 1 employs a balloon 24, suitable for us in angioplasty and/or stent delivery procedures.

In some embodiments the catheter 20 includes a shaft assembly 22, a manifold assembly 26, an inner tube 28 and an outer tube 34.

The inner tube 28 comprises a proximal end 30 and a distal end 32. The proximal end of the shaft assembly 21 extends into the manifold assembly 26. The outer tube 34 is coaxially disposed about the inner tube 28 to define an annular inflation lumen 37 in communication with the balloon 24.

The balloon 24 includes a balloon body portion 36 with a proximal balloon waist 38 and a distal balloon waist 40. The proximal balloon waist 38 is connected to the outer tube 34 near its distal end by means of an adhesive 44. The distal balloon waist 40 is connected to the inner tube 28 near its distal end 32 such that the interior of the balloon 46 is in fluid communication with the annular inflation lumen 37.

In some embodiments the catheter 20 is provided one or more radiopaque bands, markings or indicia. A radiopaque marker band 50 is engaged to the inner tube 28 or other region of the catheter 20 by any suitable engagement mechanism, such as including, but not limited to: mechanical engagement (friction fit, etc.) chemical engagement (such as by adhesive welding using cyanoacrylate or other adhesive material), heat welding, etc.

The inner tube 28 defines a guide wire lumen 54 which provides a passage for a guide wire (not shown). The outer tube 34 defines an annular inflation lumen 37 which is in fluid communication with the interior of the balloon 46.

A distal portion of the inner tube 28, which extends distally beyond the balloon 24 is provided with, or comprises a tip 110.

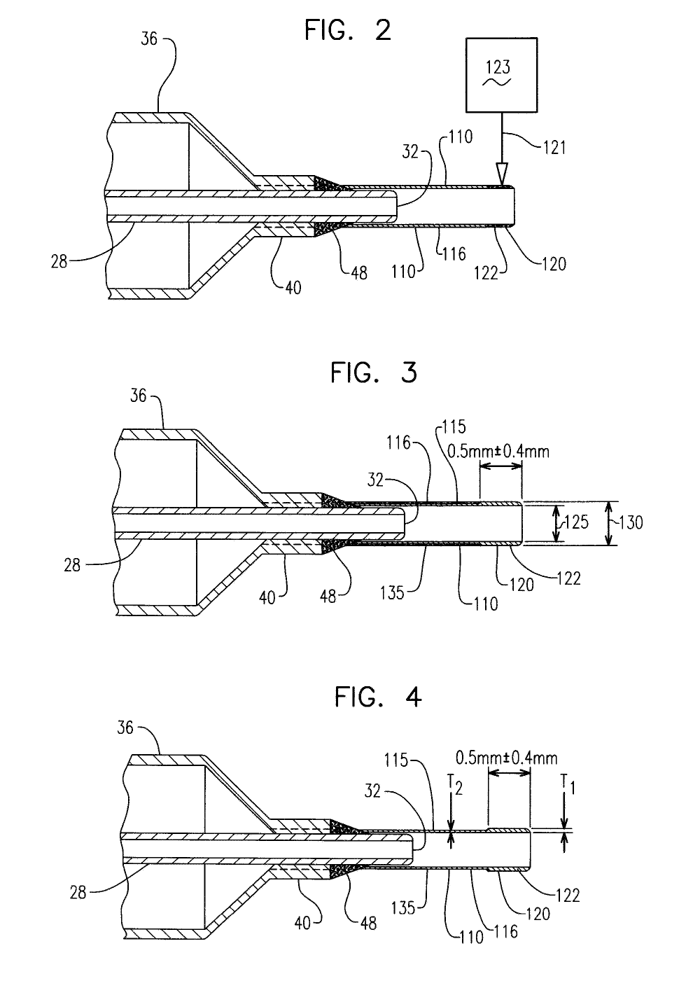

Referring now to FIG. 2, a partial cross-sectional view of the distal end of an embodiment of the invention is shown.

As indicated catheter tip 110 extends beyond the distal end 32 of the inner tube 28, the catheter tip 110 has a proximal region 115 and distal region 120. The distal region 120 comprises a distal region material 122 and the proximal region 115 comprises a proximal region material 116. The distal region material 122 has a number of modifiable physical characteristics, including hardness, crystallinity, and thickness. The distal region material 122 may be made of a variety of materials, including semi-crystalline polymers. A non-limiting list of semi-crystalline polymers include the following: nylon, polyether bloc amides like that sold under the trademark PEBAX®, combinations of nylon and polyether bloc amides, and nanocomposites.

In some embodiments of the invention the catheter tip 110 is formed by modifying one or more characteristic of the tip's material to provide for improved crossability.

In the embodiment shown in FIG. 2, at the distal region 120, heat energy (as indicated by arrow 121) is applied to the distal region 120 of the catheter tip 110. For example, a low energy laser beam could be applied without melting the tip. A non-limiting list of examples of energy sources which could be used in the following: CO₂ lasers, diode lasers, yttrium aluminum garnet (YAG) lasers, TZF fields, and UV light. Through the selective application of heat to the distal region 120, one or more of the modifiable physical characteristics of the distal region 120, of the catheter tip 110, will be modified. That is, after application of the heat energy, the distal region material 122 will become harder, more crystallized, and/or its thickness may by altered relative to the characteristics of the proximal region material 116. For example, the hardness of the distal region material 122 will become greater than the hardness of the proximal region material 116 after application of the heat energy. In addition or alternatively, the crystallinity of the distal region material 122 will become greater than the crystallinity of the proximal region material 116 after application of the heat energy.

The heat energy 121 may be supplied by any of a variety of heat sources, indicated by element 123. In the embodiment shown in FIG. 2, heat source 123 is a laser. Alternative heat sources include but are not limited to: chemical reaction, a convection source, a conductive source, etc.

In at least one embodiment, wherein the crystallinity of the distal region 120 has been modified, the crystallized area can extend along the entire length of the tip or only a portion of the tip. If only extending along a portion of the tip, the crystallized area may extend as little as approximately 0.05 mm, but the desired range of the crystallized area is 0.5 mm±0.4 mm.

In some embodiments, the proximal region material 116 is the same as the distal region material 122 (before the application of the heat energy). In some embodiments, the proximal region material 116 is different than the distal region material 122.

At least one embodiment of the invention is shown in FIGS. 3 and 4. In the embodiment shown, catheter tip 110 has an inner diameter 125 and an outer diameter 130, the inner and outer diameters defining a thickness therebetween. In manufacturing the tip 110 shown, a portion of the material of the outer diameter 130 of the proximal region 115 is ablated. Because the ablated region 135 has a reduced outer diameter, its thickness is necessarily decreased. Thus, after ablation, the distal region 120 has a greater thickness (T1) than the proximal region adjacent thereto (T2), as depicted in FIG. 4.

In some embodiments, the proximal region may comprise a second proximal region material having an inner diameter and an outer diameter defining a thickness therebetween. The step of ablating may further include ablating at least a portion of the outer diameter of the second proximal region material.

Ablation can be achieved through a number of methods, such as energy ablation (laser or thermal), mechanical ablation (cutting, etching, etc.), or chemical ablation.

In one embodiment, the distal region 120 of the catheter tip 110 with increased thickness can extend along the entire length of the tip or only a portion of the tip. If only extending along a portion of the tip, the region with increased thickness may extend as little as approximately 0.05 mm, but the desired range of the increased thickness region is 0.5 mm±0.4 mm.

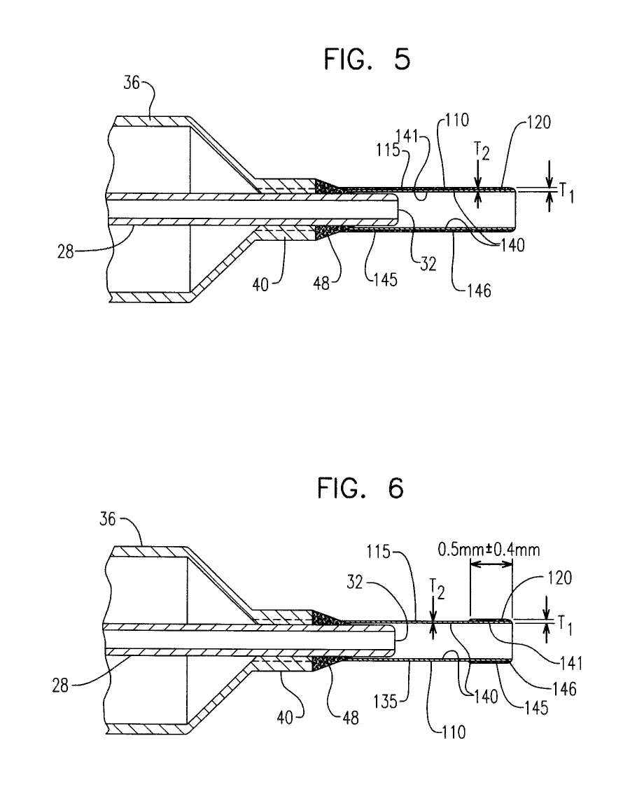

At least one embodiment of the invention is shown in FIGS. 5 and 6. In the embodiment shown in FIGS. 5 and 6, the catheter tip 110 is manufactured to have a first layer 140 comprising a first material 141, and a second layer 145 comprising a second material 146. In assembling the tip 110, the second layer 145 is concentrically disposed about the first layer 140. The first material 141 has a first durometer value and the second material 146 has a second durometer value, the second durometer value being greater than the first durometer value.

The catheter tip 110 has at least a proximal region 115 and a distal region 120. The proximal region 115 has a proximal region thickness T2 and the distal region 120 has a distal region thickness T1. A flexible, durable catheter tip is formed by ablating at least a portion of the second material 146 of at least a portion of the proximal region 115 so that the thickness T2 of the proximal region is less than the thickness T1 of the distal region 120.

Ablation can be achieved through a number of methods, such as energy ablation (laser or thermal), mechanical ablation (cutting, etching, etc.), or chemical ablation. Ablation, grinding, heating or other manufacturing processes can be performed before or after the catheter tip 110 has been engaged to the inner tube 28.

As shown in FIG. 6, only a portion of the harder second layer 145 remains after ablation. FIG. 6 shows both the softer first layer 140 and harder second layer 145 remaining at the distal region 120 of the catheter tip 110, but the hard second layer 145 is at least partially removed from the proximal region 115.

In one embodiment, the distal region 120 with the harder second layer 145 remaining can extend along the entire length of the tip or only a portion of the tip. If only extending along a portion of the tip, the distal region with the harder second layer may extend as little as approximately 0.05 mm, but the desired range is 0.5 mm±0.4 mm.

In some embodiments, the step of ablation further includes ablating at least a portion of the second material of the distal region 120. In other embodiments, the step of ablation further includes ablating most, but not all, of the second material from the proximal region. In at least one embodiment, the step of ablation further includes ablating not only the second layer 145 of the proximal region 115, but also at least a portion of the first material 141 of the proximal region 115.

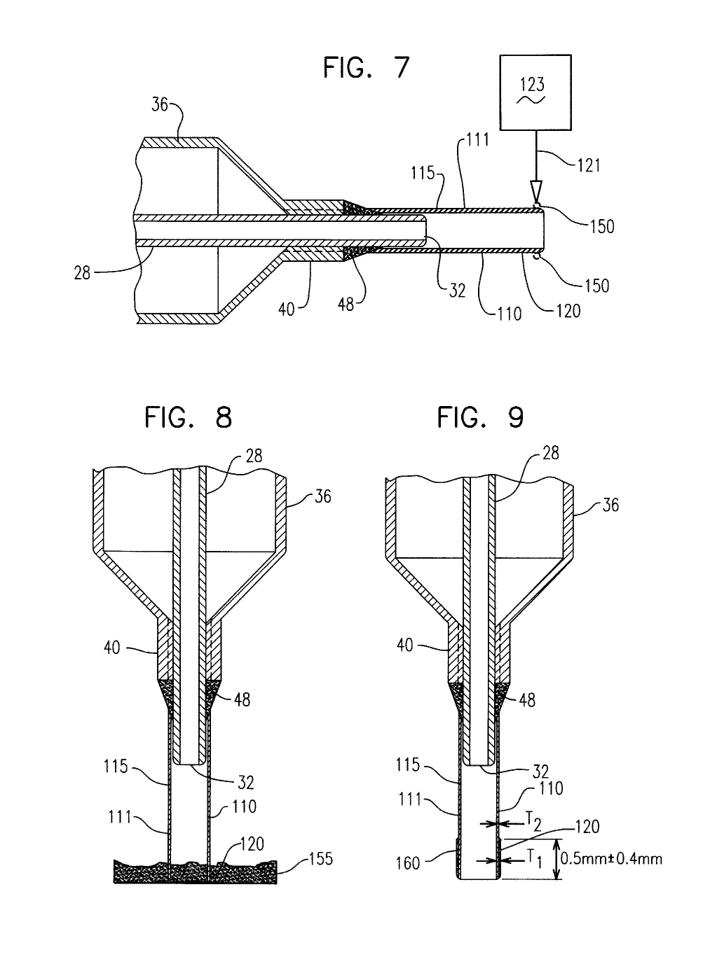

In at least one embodiment of the invention is shown in FIG. 7. The catheter tip 110 comprises a tip material, the tip material having a proximal region 115 and a distal region 120. The tip 110 includes a first tip material 111 and a curable second material 150 which is disposed about at least a portion of the first tip material.

In some embodiments the second material 150 is placed only along portions of the circumference of the tubular catheter tip 110 at its distal region 120. Also, one or more beads of second material 150 can be placed along the circumference of tubular catheter tip 110 at specific locations.

In at least one embodiment the second material 150 is cured through the application of heat energy 121. Heat energy may be provided by a number of heat sources 123, such as a laser. Also, the curable adhesive can be cured by ultraviolet light exposure, such as a polyurethane adhesive, or an acrylated urethane like that sold under the trademark LOCTITE® 3311.

In addition to ultraviolet light, curable coating systems also include, but are not limited to, electron beam (ES), peroxide, polyurethane, two-part epoxy/amine, and thermal and room temperature-cured high-performance coating systems.

Examples of low volatile organic compound (VOC) materials that can be used in such curable coating systems include, but are not limited to, the following: monomers, acrylate functionalized polymers, oligomers, photoinitiators, styrene maleic anhydride resins, hydroxyl-terminated polybutadiene resins, and functionalized polybutadiene resins and hydrocarbon resins. A person of skill in the art would recognize that there are number of alternative hardenable materials available that harden after application of pressure or heat, or after the passage of time.

In an embodiment of the invention shown in FIGS. 8 and 9, the catheter tip 110 comprises a tip material 111, the tip material having a proximal region 115 and a distal region 120. The tip material has an initial hardness and an initial crystallinity. In the embodiment of FIG. 8, the flexible, durable catheter tip 110 is formed by dipping the distal region 120 into a chemical bath 155. Of course, although FIG. 8 depicts a chemical bath 155, one of skill in the art would recognize that there are a number of alternative methods for applying a coating to the distal region 120 catheter tip 110. The chemical could be a curable adhesive that hardens after ultraviolet light exposure. A person of skill in the art would recognize that there are number of alternative chemicals available that harden after application of pressure or heat, or after the passage of time. Another method of applying a coating includes using polymers in solution and then dipping the catheter tip in the solution.

FIG. 9 depicts the present embodiment, after the chemical material has been applied. The distal region 120 with chemical 160 is shown in FIG. 9 with a thickness T1 greater than the thickness T2 of the proximal region 115. In one embodiment, the distal region 120, with increased thickness, can extend along the entire length of the tip or only a portion of the tip. If only extending along a portion of the tip, the distal region with increased thickness may extend as little as approximately 0.05 mm, but the desired range of is 0.5 mm±0.4 mm.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

What is claimed is:

1. A method of making a flexible catheter tip, the method comprising the steps of:
providing a catheter tip, the catheter tip having a proximal region and a distal region, the distal region comprising a distal region material, the distal region material having at least one modifiable physical characteristic selected from the group consisting of: hardness, crystallinity, thickness, and any combination thereof;
applying heat energy only to the distal region; and
changing the at least one physical characteristic of the distal region material.

2. The method according to claim 1, wherein the distal region has a length of at least 0.05 mm.

3. The method according to claim 1, wherein the heat energy is supplied by a heat source, the heat source being a laser.

4. The method according to claim 1, wherein the step of changing the at least one physical characteristic of the distal region material comprises increasing the hardness of the distal region material.

5. The method according to claim 1, wherein the proximal region comprises a proximal region material, the proximal region material being different from the distal region material.

6. The method of claim 5, wherein the step of changing the at least one physical characteristic of the distal region material comprises increasing the hardness of the distal region material such that the hardness of the distal region material is greater than a hardness of the proximal region material.

7. The method according to claim 5, wherein the step of changing the at least one physical characteristic of the distal region material comprises increasing the hardness of the distal region material such that the crystallinity of the distal region material is greater than a crystallinity of the proximal region material.

8. A method of making a flexible catheter tip, the method comprising the steps of:
providing a catheter tip, the catheter tip comprising a proximal region and a distal region, the proximal region comprising a proximal region material, the distal region comprising a distal region material, the proximal region material having an inner diameter and an outer diameter, the inner diameter and outer diameter defining a thickness therebetween; and
ablating at a least portion of the outer diameter of the proximal region material, wherein the thickness of the proximal region material is less than a thickness of the distal region material.

9. The method according to claim 8, wherein the step of ablating is performed by energy ablation, chemical ablation, mechanical ablation, or a combination thereof

10. The method according to claim 8, wherein the proximal region comprises a second proximal region material, the second proximal region material having an inner diameter and an outer diameter, the inner diameter and outer diameter of the second proximal region material defining a thickness therebetween, the step of ablating further including ablating at least a portion of the outer diameter of the second proximal region material.

11. The method according to claim 8, wherein the distal region has a length of at least 0.05 mm.

12. A method of making a flexible catheter tip, the method comprising the steps of:
providing a catheter tip, the catheter tip comprising a first layer comprising a first material and a second layer comprising a second material,
the second layer concentrically disposed about the first layer, the first material having a first durometer value, the second material having a second durometer value, the second durometer value being greater than the first durometer value,
the catheter tip having at least a proximal region and a distal region, the proximal region having a proximal region thickness, the distal region having a distal region thickness; and
ablating at least portion of the second material of at least a portion of the proximal region, the thickness of the proximal region being less than the thickness of the distal region.

13. The method according to claim 12, wherein the distal region has a length of at least 0.05 mm.

14. The method according to claim 12, wherein the step of ablating further includes ablating at least a portion of the second material of the distal region.

15. The method according to claim 12, wherein the step of ablating further includes ablating at least a portion of the first material of the proximal region.

16. A method of making a flexible catheter tip, the method comprising the steps of:
providing a catheter tip, the catheter tip constructed of a tip material having a hardness and a crystallinity, the catheter tip having a proximal region and a distal region;
dipping at least a portion of the distal region into a chemical bath; and
changing the hardness and/or crystallinity of the tip material in the distal region, such that the hardness and/or crystallinity of the distal region is different than the hardness and/or crystallinity of the proximal region.

17. The method according to claim 16, wherein in the step of changing, the hardness of the tip material in the distal region is made greater than the hardness of the tip material in the proximal region.

18. The method according to claim 16, wherein in the step of changing, the crystallinity of the tip material in the distal region is made greater than the crystallinity of the tip material in the proximal region.

19. A method of making a flexible catheter tip, the method comprising the steps of:
providing a catheter tip, the catheter tip constructed of a tip material, the catheter tip having a proximal region and a distal region;
adding a second material to the catheter tip, the second material being a curable adhesive applied to at least a portion of the distal region;
applying heat energy to the at least a portion of the distal region to cure the curable adhesive.

20. The method of claim 19 wherein the heat energy is provided by a heat source, the heat source being a laser.
